(19) United States
(12) Patent Application Publication
(10) Pub. No.: US 2017/0274128 A1
(43) Pub. Date: Sep. 28, 2017

(54) PUMPING SYSTEMS, ENDOLUMINAL DEVICES AND SYSTEMS FOR CREATING TWO-WAY BLOOD FLOW

(71) Applicants: Corrado TAMBURINO, Sant'Agata Li Battiati, CATANIA (IT); Claudia TAMBURINO, Sant'Agata Li Battiati, CATANIA (IT); Sebastiano IMME', Melilli, SIRACUSA (IT)

(72) Inventors: Corrado TAMBURINO, Sant'Agata Li Battiati, CATANIA (IT); Claudia TAMBURINO, Sant'Agata Li Battiati, CATANIA (IT); Sebastiano IMME', Melilli, SIRACUSA (IT)

(21) Appl. No.: 15/532,110

(22) PCT Filed: Dec. 14, 2015
(86) PCT No.: PCT/IB2015/059593

§ 371 Date: Jun. 1, 2017

(30) Foreign Application Priority Data
Dec. 16, 2014 (IT) BS2014A000211

(51) Int. Cl. A61M 1/12; A61M 1/10

(52) U.S. Cl. CPC A61M 1/125; A61M 1/1029; A61M 1/1037; A61M 1/1087; A61M 1/122; A61M 1/1012; A61M 2205/75; A61M 2205/32; A61M 2230/30; A61M 2230/005; A61M 2205/3365

(57) ABSTRACT

A pumping system (200) for controlling the flow of interatrial blood comprises, housed inside a container (201), a control element (30, 30′, 30″) of the interatrial blood flow. The control element comprises: at least one worm screw (31), the rotation of which creates a two-way flow of interatrial blood; or a pair of counter-rotating propellers (31′); or a pair of membranes (31″) whose deformation creates a two-way flow of interatrial blood; or a flexible structure (31″) whose change in volume within the container (201) creates a two-way flow of interatrial blood.

This invention relates to a pumping system, an endoluminal device and an endoluminal system for the treatment of heart failure, in particular for the creation of a controlled, two-way, interatrial blood flow.

The syndrome of heart failure is a common pathophysiologic state in which the heart is unable to pump blood at a level adequate to the needs of the metabolising tissues or can do so only with a high diastolic filling pressure. This clinical syndrome, which can be caused by various pathological conditions, is characterised by a low cardiac output and an increase of the intracardiac filling pressure.

It should be noted that about a third of patients with heart failure suffer from diastolic heart failure whose symptoms are due, in most cases, to an increase of pressure in the left atrium. This increase in left atrial pressure causes an increase in pulmonary venous pressure, a condition partly responsible for dyspnea. These symptoms include chronic breathlessness, a condition that worsens in a supine position, causing small changes in pulmonary venous pressure, with consequent aggravation of the symptoms. This condition may progress to pulmonary oedema, one of the most dangerous consequences associated with the syndrome of heart failure. Pulmonary oedema is caused by the sudden increase of capillary hydrostatic pressure and accumulation of fluid with low protein content in the interstitium and pulmonary alveoli. Without proper and prompt treatment, this condition can rapidly deteriorate and eventually lead to death.

As regards the right side of the heart, pulmonary hypertension, a rare disease characterised by right ventricular failure and high right atrial pressure, can lead to the systemic venous congestion and eventually to death. One of the therapeutic strategies adopted in the late stages of pulmonary hypertension is the atrial septostomy, a percutaneous intervention aimed at reducing the high right atrial pressure by creating a hole in the native atrial septum. This procedure has been shown to be effective in increasing cardiac output, improving the symptoms of heart failure and reducing hyperactivity of the sympathetic autonomic nervous system.

Therefore, in the medical field there is a need for devices and methods for the treatment of heart failure as regards both the right and left side of the heart.

The purpose of this invention is to provide a pumping system, an endoluminal device and an endoluminal system for the treatment of heart failure that solves the problems of the known art while taking into account the needs of the sector.

In particular, the purpose of this invention is to provide a pumping system, an endoluminal device and an endoluminal system for adjusting the amount of interatrial blood flowing from left to right or from right to left, as a function of the atrial pressure.

This purpose is achieved by a pumping system according to claims 1, 7, 10 and 12, by an endoluminal device according to claim 15 and by an endoluminal system according to claim 17. The dependent claims describe preferred or advantageous embodiments of the system and device.

The characteristics and advantages of the pumping system, endoluminal device and endoluminal system according to this invention will be apparent from the following description, given by way of non-limiting example, in accordance with the accompanying figures, wherein:

FIG. 1 shows the endoluminal device for controlling the interatrial flow, according to this invention, positioned inside the patient's heart;

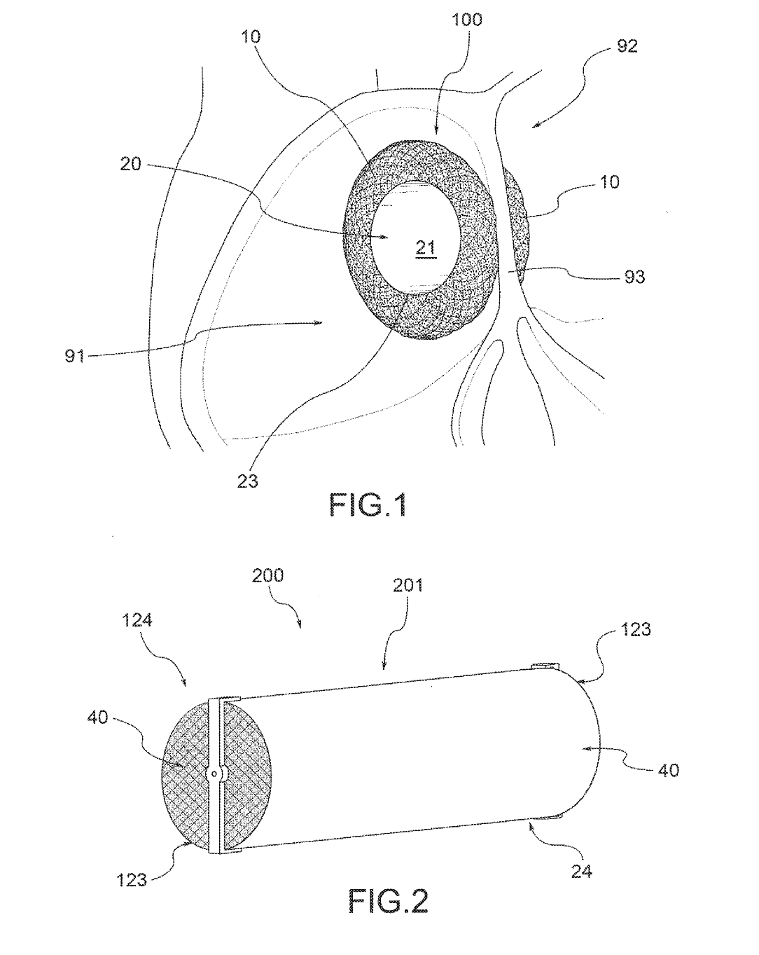

FIG. 2 shows an axonometric view of a pumping system according to this invention, according to a first embodiment variant;

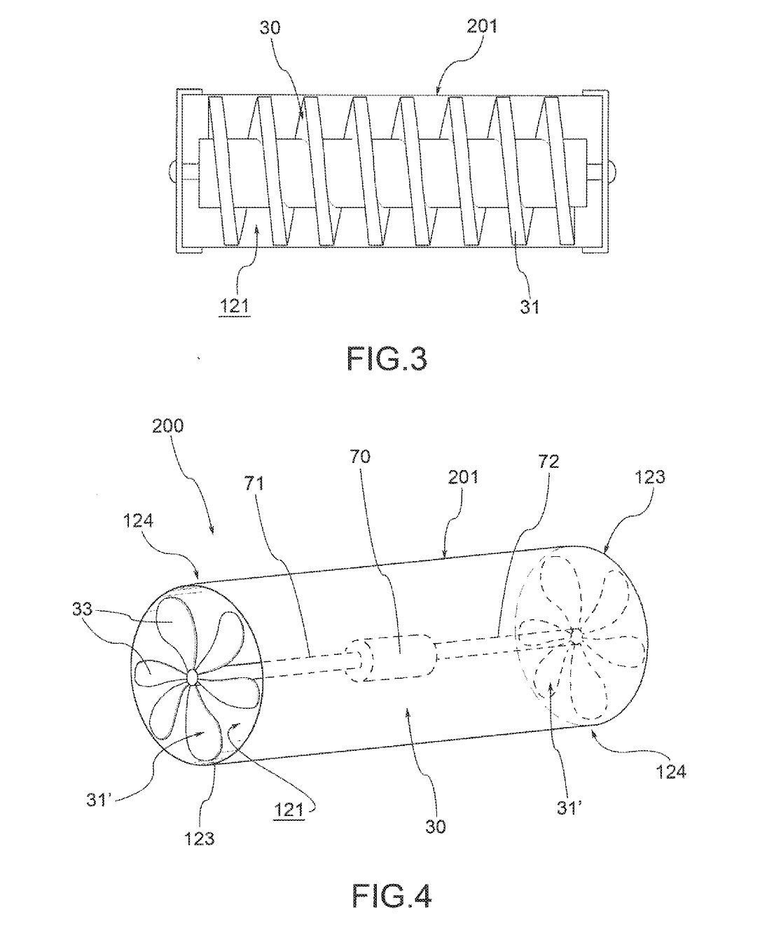

FIG. 3 shows a side view of the pumping system of FIG. 2;

FIG. 4 shows an axonometric view of a pumping system according to this invention, according to a further embodiment variant;

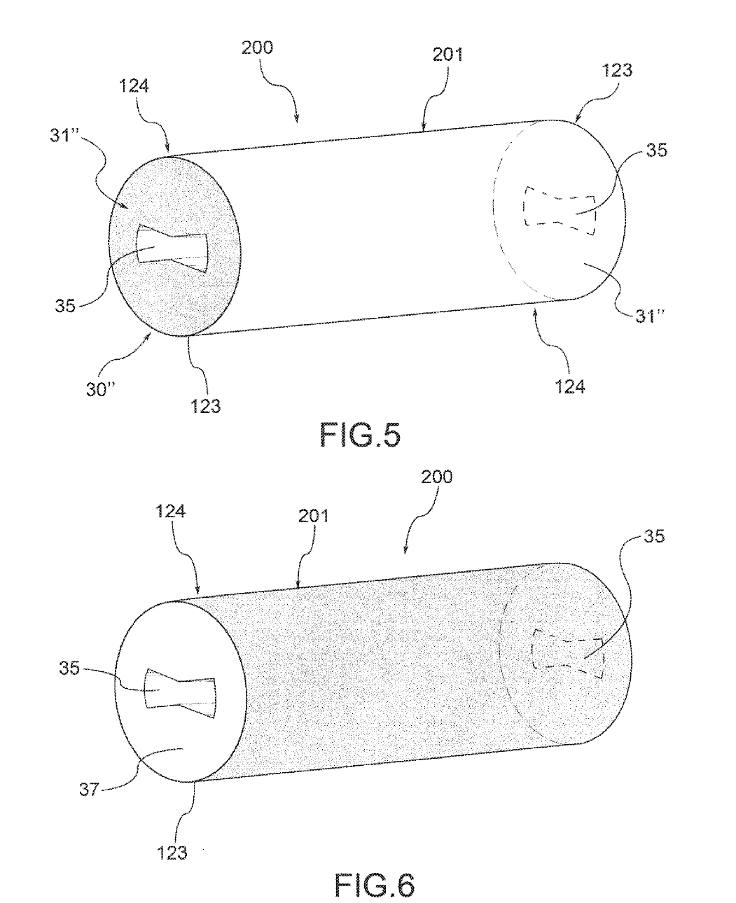

FIG. 5 shows an axonometric view of a pumping system according to this invention, according to a still further embodiment variant;

FIG. 6 shows an axonometric view of a pumping system according to this invention, according to a still further embodiment variant.

The accompanying figures, and in particular to FIG. 1, show an endoluminal device 100 for controlling the interatrial blood flow. The endoluminal device 100 is suitable to be positioned inside the patient's heart, in particular in a hole 93 present in the atrial septum or specifically created by the operating cardiologist.

The endoluminal device 100 comprises a central body 20 suitable to be positioned in a hole present in the wall or interatrial septum 93 of the heart. The central body 20 is substantially cylindrical and comprises an inner lumen 21 suitable to allow the interatrial passage of blood. The central body 20 has, in correspondence of each end, an access opening 23 to the central lumen 21. The openings 23 permit the passage of interatrial flow and the pumping of blood.

The endoluminal device 100 comprises at least one anchor 10 suitable to keep the central body 20 in position inside the patient's heart.

Preferably, the endoluminal device 100 comprises a pair of anchors 10, each fastened to one end of the central body 20.

During the release of the endoluminal device 100, a first anchor is positioned in the left atrium 91 and a second anchor is positioned in the right atrium 92, so as to keep the endoluminal device 100 in position between the right atrium 92 and the left atrium 91.

In an embodiment variant, the anchor is a mesh, a substantially open. The mesh is made of material suitable for use in a patient, such as titanium, nitinol, stainless steel, Elgiloy®, MP35NED, Vitalium™, Mobilium™, Ticonium™, Platinore™, Stellite®, tantalum, platinum or other elastic material. Alternatively, the mesh 11 is made of polymeric material, such as PTFE, UHMWPE, HDPE, polypropylene, polysulfone, or other biocompatible plastic material.

Preferably, to improve the process of endothelialisation, the mesh is provided with a covering layer made of bioabsorbable polymer, such as polylactic acid, polyglycolic acid, polycaprolactone, a combination of two or more of these bioabsorbable polymers, or is covered by a mesh of bioabsorbable fabric.

In an embodiment variant, the mesh is made using a preformed wire, folded back on itself and secured in the desired shape by, for example, welding or adhesive.

In a further embodiment variant, the mesh is made from a hollow tube, drilled to form an open mesh, for example using a laser cutter or water drill.

In an embodiment variant, the anchor is made from a plurality of flanges, which extend radially outwardly from the central body.

Preferably, the endoluminal device 100 comprises a plurality of radiopaque markers, used to easily identify the end of the endoluminal device 100 with non-invasive techniques, such as X-rays or ultrasound, during or after the procedure. Preferably, the markers are applied to the anchors.

The endoluminal device 100 comprises a pumping system 200 suitable to create a controlled two-way flow of interatrial blood.

The pumping system 200 can be fixed in the inner lumen 21 of the central body 20 of the endoluminal device 100.

The pumping system 200 comprises a container 201 in which are housed at least one control element 30,30′,30″ of the flow and activation means for the activation of the control element 30,30′,30″.

Preferably, the container 201 is made of metallic material or of biocompatible polymeric material.

Preferably, the container 201 is equipped with a coupling system that allows the positioning of the pumping system 200 in the central body 20 of the endoluminal device 100.

It is important that the control element 30 is inserted flush into the container 201, and that the tolerances are minimal (while ensuring the rotation of the control element 30) so as to reduce to a minimum the risk of haemolysis of the erythrocytes and activation the complement, in other words it is important to avoid crushing of red blood cells that, otherwise, breaking trigger an inflammatory response of the vascular system.

The container 201 has, in correspondence of each end 124, an access opening 123 to the central lumen 121. The openings 123 permit the passage of interatrial flow and the pumping of blood.

In the embodiment variant shown in FIGS. 2 and 3, the control element 30 comprises at least one worm screw 31, housed in the container 201, driven by an electric motor (not shown), preferably of the brushless, three-phase electric type.

In the variant of FIG. 3, wherein the control element 30 comprises a worm screw 31, the electric motor can be housed in the front or rear position with respect to the worm screw 31, in such a way that an extension, respectively front or rear of the rotary axis of the motor, is mechanically connected to the worm screw 31.

In an embodiment variant not shown, the control element 30 comprises a pair of worm screws. Preferably, the electric motor is housed in a central position with respect to the control element 30, in such a way that a front extension of the rotary axis of the motor incorporates a front screw and a rear extension of the rotary axis of the motor incorporates a rear screw.

The control element 30 extends axially inside the container 201, and preferably extends for the entire length of the container body 201.

The container 201 is provided with at least one filter 40 positioned in correspondence of the aperture 123. Preferably, the filter 40 completely covers the opening 123.

Preferably, the container 201 is provided with two filters 40, one for each opening 123.

The filter 40 is made of biocompatible material.

The filter 40 has a porosity such as to allow the passage of the figured elements of the blood, which is to say the corpuscular part of the blood, but at the same time to prevent the passage of micro-clots or aggregates of platelets. Preferably, the filter 40 has a porosity of about 10 μ.

In a further embodiment variant, shown in FIG. 4, the control element 30′ comprises at least one propeller 31′, preferably house in correspondence of one end 124 of the container 201. Preferably, the propeller 31′ is housed in correspondence of the access opening 123 to the central lumen 121.

The control element 30′ is actuated by at least one motor 70, preferably of the brushless, three-phase electric type. The motor 70 is fixed, preferably inside the container 201, by means of specific supports. The electric motor 70 provides power to the propeller 31′, which rotates so as to establish a blood flow that, depending on the rotation imposed by the motor 70, can be two-way.

Preferably, the control element 30′ comprises a pair of propellers 31′, each positioned in correspondence of an opening 123. The electric motor 70 provides power to the propellers 31′ that preferably rotate in counter-rotation so as to establish a blood flow that, depending on the rotation imposed by the motor 70, can be two-way. Advantageously, the presence of a pair of counter-rotating propellers 31′ allows cancelling the moment of inertia of the propellers themselves so as to avoid trauma to biological structures during the steps of changing speed or turning the control element 30′ on or off.

In the embodiment variant shown in FIG. 4, the motor 70 is positioned axially relative to the container 201, in a central position relative to the two propellers 31′. The motor 70 comprises a front extension 71 of the rotary axis of the motor fixed to a first propeller 31′ and a rear extension 72 of the rotary axis of the motor fixed to a second propeller 31′.

In further variants not shown, the motor is positioned laterally inside the container 201.

Preferably, the propeller 31′ comprises a plurality of blades 33. The propeller 31′ comprises at least five blades 33. Preferably, the propeller 31′ comprises at least ten blades 33, preferably twelve. Advantageously, the propeller 31′ comprises a large number of blades 33 so that the pitch between one blade and the next is minimal. Such construction allows to reducing the rotation speed of the control element 30′ and minimising the trauma exerted on the figured blood elements.

In the embodiment variants shown in FIGS. 2 to 4, the control element 30,30′ is rotatably engaged with respect to the container 201 and, when inserted inside the central body 20, with respect to the endoluminal device 100. The rotation of the control element 30,30′ allows creating a controlled two-way flow of interatrial blood. In particular, the rotation of the control element 30,30′ allows obtaining a forced two-way flow of interatrial blood from right to left or from left to right, depending on the hemodynamic needs. In particular, the control element 30,30′ operates based on the pressure gradient between the right atrium and left atrium, due to increased pressure in the atrium in which a decompensation is present. Therefore, the control element 30,30′ allows mitigating this pressure gradient between the two cavities.

In a further embodiment variant, shown in FIGS. 5 and 6, the control element 30″ comprises at least one flexible structure activated by suitable activation means.

Preferably, the flexible structure is a membrane 31″ positioned in correspondence of an end 124 of the container 201. Preferably, the membrane 31″ is housed in correspondence of the access opening 123 to the central lumen 121.

The membrane 31″ is equipped with a one-way valve 35 that allows the passage of interatrial flow.

In the embodiment variant of FIG. 5, the control element 30″ comprises a pair of membranes 31″, each positioned in correspondence of an opening 23 of the container 201.

Preferably, the activation means comprise an electromagnet, for example a solenoid wound on soft iron, activated by an alternating electric current and suitable to move at least one magnet connected to the flexible structure 31″. The activation means activate the control element 30″ and, in particular, determine a deformation of the flexible structure 31″ so as to establish a controllable two-way flow. In this case, the frequency of the electricity flowing in the electromagnet influences the volume of the two-way flow.

In a further embodiment variant, shown in FIG. 6, the flexible structure 31″ is positioned inside the container 201, in correspondence of the inner circumference of the container 201.

A pair of membranes 37 is positioned at the closure of the openings 123 of the container 201; each membrane 37″ is equipped with a one-way valve 35 that allows the passage of interatrial flow. The activation of the flexible membrane 31″ involves a change of volume inside the container 201: this volume change allows a variation of interatrial flow, which passes through the container 201 by means of the two one-way valves 35 placed on the membranes 37.

In a variant, the flexible structure 31″ is a balloon.

In a further variant, flexible structure 31″ is composed of fibres able to vary their length upon the transit of electricity.

The control element 30,30′,30″ is made from biocompatible materials with a particularly smooth surface, and which do not have anionic charges capable of activating the coagulative cascade (such as the titanium dioxide).

Preferably, the endoluminal device 100 and/or the container 201 is provided with, for example, in correspondence of each end 24,124, at least one sensor for monitoring hemodynamic parameters, such as the pressure of the two atria or the saturation of blood oxygen.

The data collected by the sensor, as well as the current for the activation of the motor, are conveyed respectively via a data transmission device and an energy transmission device. Said devices are for example cables inside of which there are at least six conductors. These cables are made of biocompatible materials and are coated with further materials that allow fast endothelialisation, such as PTFE or polylactic acid.

All the variants the pumping element 200 shown in FIGS. 2 to 6, wherein the control element 30 is housed in the container 201, can be directly implanted in the atrial septum, or in other parts of the human body, without the aid of the endoluminal device 100. In this embodiment, the pumping system 200 allows the creation of a two-way flow that can be exploited, in addition to in the interatrial septum, also as a blood pumping system in general, suitable to be implanted in other parts of the body, for example as a ventricular assist system.

The endoluminal device 100 and/or the pumping system 200 according to this invention is adjusted by a control unit suitable to process information relating to the pressure and to the control element 30,30′,30″ (for example speed and direction of rotation) and to adjust the two-way interatrial blood flow. Preferably, the control unit is a pulse generator (for example of the pacemaker type) controllable for example by means of cable or wireless or remotely. The information detected by the sensors (such as the pressure) are sent to the control unit that, by means of specific algorithms, calculates and defines the rotation speed of the control element 30,30′ and the direction of rotation, or speed deformation of the control element 30″.

The control unit is positioned for example in a special pocket created under the skin of the patient, in the upper part of the chest.

The control unit is controlled via software, customisable and interactive, controlled for example via a transceiver, cell phone, or other telemetric-type instrumentation.

By means of specific software programs, it is possible to access information related to the endoluminal device 100 and/or the pumping system 200 (interatrial pressure, rotation speed of the control element, direction of rotation of the control element) and make changes to the operating algorithms so as to adjust the two-way interatrial flow based on clinical needs.

The endoluminal device 100 and/or the pumping system 200 according to this invention is suitable to be implanted in the ascending aorta, in particular in the outflow region of the left ventricle, between the aortic valve and a window between the left atrium and ascending aorta obtainable after transseptal puncture (right-left) and subsequent puncture between left atrium and ascending aorta. In these cases the endoluminal device has the function of supporting, through direct pumping from the left atrium to the ascending aorta, the left circle in case of temporary deficit of the pumping function of the left ventricle. The endoluminal device also has the function of VAD (Ventricular Assist Device) usable as a bridge while waiting for a heart transplant, and then find application in the case of left ventricular failure.

The endoluminal device 100 is adapted to be compressed (or collapsible) to a substantially cylindrical shape to be fitted onto a delivery catheter for positioning in the patient's heart.

This invention also relates to an endoluminal system for the treatment of heart failure, and in particular for the control of interatrial blood for the creation of a controlled two-way flow. This system comprises:
an endoluminal device 100 and/or a pumping system 200 comprising at least one control element 30,30′,30″ suitable to create a controlled two-way flow of interatrial blood;
a release catheter for the transport and positioning of the endoluminal device 100 and/or the pumping system 200 in the patient;
an actuation device (for example a motor or an electromagnet) for the actuation of the control element;
a device for transmitting energy to power the actuation device;
at least one sensor for monitoring the haemodynamic parameters;
a control unit for processing the information gathered by the sensor and for the regulation of the two-way interatrial blood flow;
a data transmission device, to transmit data between the sensor, control unit and actuation device.

Innovatively, a pumping system, an endoluminal device and an endoluminal system for the control of interatrial blood suitable to create a controlled two-way flow, according to this invention, allows reducing the high pressure in the right and in the left side of the heart, thereby relieving the symptoms of heart failure and ventricular overload.

Advantageously, a pumping system, an endoluminal device and an endoluminal system, according to this invention, allows the control and adjustment of the amount of blood that flows from left to right or from right to left depending on the increase of the atrial pressure above a set maximum limit, in order to reduce the rate and severity of pulmonary oedema or of right ventricular failure.

It is clear that one skilled in the art may make changes to the endoluminal device and the endoluminal system described above, all contained within the scope of protection defined by the following claims.

1-17. (canceled)

18. A pumping system for the creation of a controlled two-way flow of interatrial blood, comprising:
a container having an inner lumen suitable for allowing passage of interatrial blood and, at each end, an access opening to a central lumen; and
a control element, engaged in a rotatable manner to the container, suitable for controlling flow of interatrial blood;
wherein the control element comprises at least a worm screw, housed inside the container, the rotation of which creates a two-way flow of interatrial blood.

19. The pumping system of claim 18, wherein the control element comprises a pair of worm screws.

20. The pumping system of claim 18, wherein the control element is driven by an electric motor housed inside the container.

21. The pumping system of claim 18, wherein the container comprises metal or biocompatible polymer material.

22. The pumping system of claim 18, wherein the container is provided, at each opening, with a filter.

23. The pumping system of claim 22, wherein the filter comprises biocompatible material and has a porosity of about 10 μ.

24. A pumping system for the creation of a controlled two-way flow of interatrial blood, comprising:
a container having an inner lumen suitable for allowing passage of interatrial blood and, at each end, an access opening to a central lumen; and
a control element engaged in a rotatable manner to a central body, suitable for controlling flow of interatrial blood;
wherein the control element comprises a pair of counter-rotating propellers, each housed in an opening, the rotation of which creates a two-way flow of interatrial blood, and wherein each propeller comprises a plurality of blades.

25. The pumping system of claim 24, wherein each propeller comprises at least 5 blades.

26. The pumping system of claim 25, wherein each propeller comprises between 10 and 12 blades, inclusive.

27. The pumping system of claim 24, wherein the control element is driven by an electric motor.

28. A pumping system for the creation of a controlled two-way flow of interatrial blood, comprising:
a container having an inner lumen suitable for allowing passage of interatrial blood and, at each end, an access opening to a central lumen; and
a control element, engaged to the container, suitable for controlling flow of interatrial blood;
wherein the control element comprises a pair of membranes, each positioned at an opening and each fitted with a one-way valve, the deformation of which creates a two-way flow of interatrial blood.

29. The pumping system of claim 28, wherein the control element is driven by an electromagnet activated by an alternating electric current and suitable for moving at least one pair of magnets, each connected to a membrane.

30. A pumping system for the creation of a controlled two-way flow of interatrial blood, comprising:
a container having an inner lumen suitable for allowing passage of interatrial blood and, at each end, an access opening to a central lumen; and
a control element, engaged to the container, suitable for controlling flow of interatrial blood;
wherein the control element comprises a flexible structure positioned inside the container, and a pair of membranes provided at the openings of the container, each fitted with a one-way valve, wherein the actuation of a flexible membrane leads to a change in volume inside the container and the creation of a two-way flow of interatrial blood.

31. The pumping system of claim 30, wherein the flexible structure is composed of fibres able to vary their length upon the transit of electricity.

32. The pumping system of claim 30, wherein the flexible structure is a balloon.

33. An endoluminal device for controlling the flow of interatrial blood, comprising:
a central body having an inner lumen suitable for allowing passage of interatrial blood and, at each end, an access opening to a central lumen;
a pumping system of claim 18, and wherein the container is fitted with a coupling system for its attachment inside the central body.

34. The endoluminal device of claim 33, provided, at each end of the central body, with an anchor suitable for keeping the device in position.

35. An endoluminal system for controlling flow of interatrial blood, comprising:
an endoluminal device of claim 34;
a positioning catheter for transport and positioning of the endoluminal device inside a patient;
an actuation device for actuation of a control element;
a device for transmitting energy to power the actuation device;
at least one sensor for monitoring haemodynamic parameters;
a control unit for processing information gathered by the sensor and for regulating a two-way interatrial blood flow;
a data transmission device, to transmit data between the sensor, control unit and actuation device.
